(19) United States
(12) Patent Application Publication
(10) Pub. No.: US 2025/0331728 A1
(43) Pub. Date: Oct. 30, 2025

(54) METHODS AND SYSTEMS FOR CARDIO-RESPIRATORY HEALTH MONITORING

(71) Applicants: George SHAKER, Waterloo (CA); Serene ABU-SARDANAH, Toronto (CA)

(72) Inventors: George SHAKER, Waterloo (CA); Serene ABU-SARDANAH, Toronto (CA)

(21) Appl. No.: 19/188,273

(22) Filed: Apr. 24, 2025

Related U.S. Application Data
(60) Provisional application No. 63/638,085, filed on Apr. 24, 2024.

(51) Int. Cl. A61B 5/024; A61B 5/00; A61B 5/05

(52) U.S. Cl. CPC A61B 5/02444; A61B 5/05; A61B 5/7257

(57) ABSTRACT

The disclosure is directed at systems and methods of health monitoring. The disclosure includes a radar system that has a set of transmitters for transmitting electromagnetic waves at an individual of interest and a set of receivers for receiving reflected and/or deflected electromagnetic waves. The received reflected and/or deflected electromagnetic waves are then processed to determine or generate health monitoring signals.

### CROSS-REFERENCE TO OTHER APPLICATIONS

The disclosure claims priority from U.S. Provisional Application No. 63/638,085 filed Apr. 24, 2024, which is hereby incorporated by reference.

### FIELD

The disclosure is generally directed at health monitoring and, more specifically, at methods and systems for cardio-respiratory health monitoring.

### BACKGROUND

Cardiovascular and respiratory diseases are significant global health concerns, causing high rates of illness and death. According to the World Health Organization (WHO), cardiovascular diseases (CVD) are a leading cause of global deaths, accounting for approximately 17.9 million deaths annually. Similarly, respiratory diseases like chronic obstructive pulmonary disease (COPD) and asthma affect millions of people, leading to high healthcare costs and reduced quality of life. Monitoring cardiopulmonary health is especially important as studies show decreased cardiovascular and pulmonary performance post-infection, especially among older adults and those with comorbidities. With the rise of cardiovascular and respiratory diseases worldwide, monitoring one's cardiac health and pulmonary health are crucial for an individual's overall well-being.

Traditional methods like electrocardiogram (ECG) and spirometry have limitations, such as complexity, skin irritations, lack of continuous monitoring, and the need for patient cooperation.

Therefore, there is provided novel methods and systems for cardio-respiratory health monitoring.

### SUMMARY

The disclosure is directed at a system and method for cardio-respiratory health monitoring. In one embodiment, the disclosure includes a radar component operating in a near-field environment that is located proximate an individual of interest. The radar component may be integrated within a wearable; may be integrated within furniture or may be mounted to a wall or structure that an individual is passing by.

In some embodiments, an advantage of the disclosure is the provision of information about the displacement waveforms associated with the movement of an individual's chest during respiration and a cardiac cycle. Additionally, the disclosure can measure health signals such as, but not limited to, respiratory rate (RR), heart rate (HR), and heart rate variability (HRV) of the individual.

In another embodiment, the disclosure is directed at a radar-based vital signal monitoring system that can track biological data such as, but not limited to, breathing rate, heart rate, and heartbeat waveform in a continuous, non-obtrusive manner.

In one aspect of the disclosure, there is provided a method for health monitoring including transmitting electromagnetic waves at a target of interest; receiving reflected electromagnetic waves from the target of interest; and processing the reflected electromagnetic waves to determine health monitoring signals; wherein processing the reflected electromagnetic waves includes performing a target bin calculation and extracting waveforms from the reflected electromagnetic waves.

In another aspect, performing a target bin calculation includes generating a radar data cube based on the reflected electromagnetic waves; removing clutter from the radar data cube; and applying a range Fast Fourier transform (FFT) to the radar data cube. In a further aspect, before generating a radar data cube, processing the down-converting the reflected electromagnetic waves. In yet another aspect, extracting waveforms from the reflected electromagnetic waves includes applying a range FFT to the radar data cube; selecting a target range from the target bin calculation; extracting phase from the radar data cube; unwrapping phase from the radar data cube; and transforming radar data cube to a displacement array to generate heart rate.

In another aspect of the disclosure, there is provided a system for health monitoring including a set of transmitters for transmitting electromagnetic waves toward an individual of interest; a set of receivers for receiving electromagnetic waves reflected or deflected off the individual of interest; and a processor for processing the reflected or deflected electromagnetic waves to determine a target bin calculation and to extract waveforms from the reflected or deflected electromagnetic waves.

### BRIEF DESCRIPTION OF THE DRAWINGS

Embodiments of the present disclosure will now be described, by way of example only, with reference to the attached Figures.

FIG. 1 is a schematic diagram of a system for health monitoring in an operating environment;

FIG. 2 is a flowchart showing a method of health monitoring;

FIG. 3 is a flowchart showing a method of processing received signal for determining health monitoring signals;

FIG. 4a is a flowchart showing a method of determining cardio-respiratory health monitoring signals;

FIG. 4b is a schematic diagram of a radar data cube showing matrix dimensions;

FIG. 4c shows a range Fast Fourier Transform operation on a radar cube dimension;

FIG. 4d is a diagram showing target position at 0.03 for one of the K frames;

FIG. 4e is a graph showing a sample spectrum showing different frequency components during vital signs extraction with respiratory rate of 10 breaths/minute and heart rate of 70 beats/minute;

FIGS. 4f and 4g are schematic diagrams of phase continuities addressed through phase unwrapping for phase discontinuity greater than π (FIG. 4f) and phase discontinuity great than −π (FIG. 4g); and

FIG. 4h are graphs showing examples of phrase unwrapping with and without up-sampling.

### DETAILED DESCRIPTION

The disclosure is directed at methods and system for health monitoring, such as, but not limited to, cardio-respiratory health monitoring. In some embodiments, the disclosure may be implemented or integrated within a wearable equipment or component. In other embodiments, the disclosure may be implemented within furniture or mounted to a wall within a skin contact distance from an individual.

In one embodiment, the disclosure includes a Frequency Modulated Continuous Wave (FMCW) radar component operating in a near-field environment with respect to the individual of interest. In the following description, the individual of interest, or individual, represents the person that is experiencing cardio-respiratory health monitoring. In one specific embodiment, the radar component operates at about 60 GHz which allows for the continuous extraction of various vital signs related to cardio-respiratory activity.

An advantage of the disclosure is the provision of information relating to the displacement waveforms associated with the movement of the chest during respiration and a cardiac cycle. Additionally, the disclosure may measure the respiratory rate (RR), heart rate (HR), and heart rate variability (HRV) of the individual as part of the cardio-respiratory health monitoring. In other embodiments, another advantage of the disclosure includes, but is not limited to, continuous and real-time capture of vital cardio-respiratory health monitoring signal rates and waveforms.

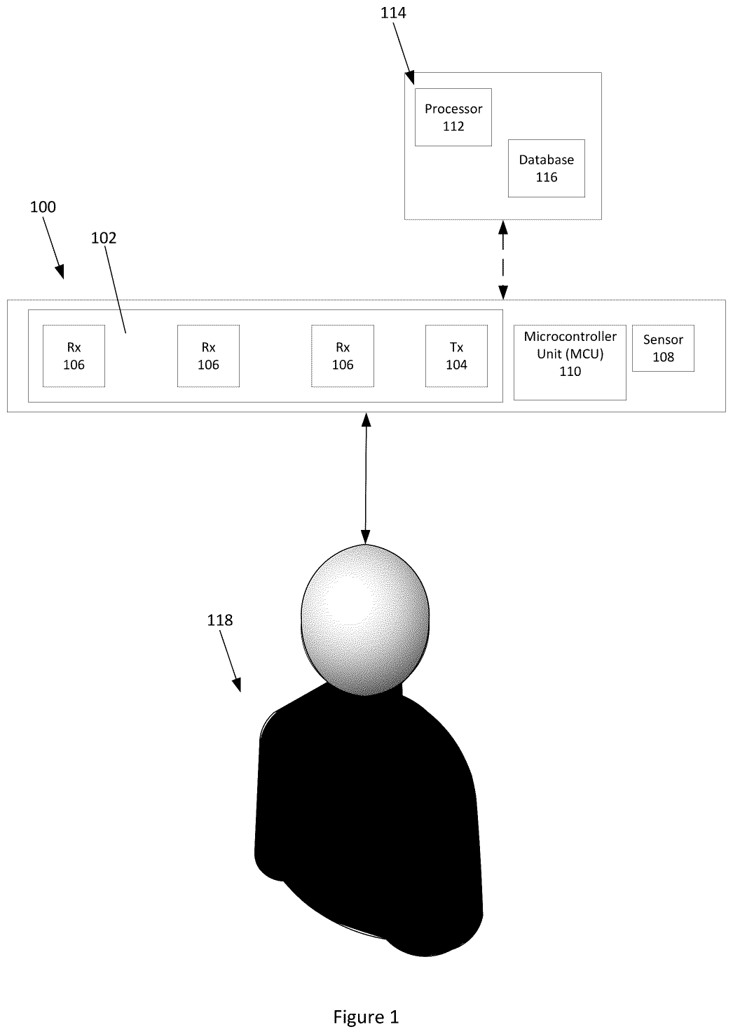

Turning to FIG. 1, a schematic diagram of a cardio-respiratory health monitoring system in its operational environment is shown. It will be understood that FIG. 1 is not shown to scale in order to more clearly identify components of the health monitoring system.

The health monitoring system 100 includes a radar component, radar module or radar system 102 that includes a set of transmitters 104 and a set of receivers 106 for transmitting electromagnetic signals and for receiving reflected electromagnetic signals, respectively. In the current embodiment, the radar component 102 includes one transmitter 104 and three (3) receivers 106, however, any number of transmitters and/or receivers may be chosen.

The system 100 further includes a sensor component 108 and a microcontroller unit (MCU) 110. In some embodiments, the sensor component 108 may be integrated with or may be the radar component 102. In some embodiments, the MCU 110 may be a printed circuit board (PCB).

Health monitoring system 100 may include, or may be connected to, a processor 112 (which in the current embodiment is located within a user communication device 114) for processing the reflected electromagnetic signals received by health monitoring system 100 to generate results relating to health monitoring. These results may be displayed to the individual or a health practitioner or may be stored in a database 116 for further analysis or analytics. In other embodiments, the database 116 may be used for storing results or any other digital information or data. Examples of the user communication device 114 include, but are not limited to, a Smartphone, a laptop, a server, a desktop computer, a tablet and the like. Communication between the health monitoring system 100 and the user communication device 114 is facilitated using known communication protocols. In some embodiments, the health monitoring system 100 may communicate with processor 112 wirelessly and, in other embodiments, the received reflected signals, which may also be referred to as digital data, may be transmitted via a Universal Serial Bus (USB) connection between the health monitoring system 100 and the processor 112. In some embodiments, instead of being part of a user communication device 114, the processor 112 may be a server or part of a server that stores and processes the received reflected signals and then transmits results to a predetermined destination and/or displays the results. The results may include, but are not limited to, health bio-markers, cardio-vascular measurements, cardio-respiratory measurements and the like.

In use, the system 100 is placed within a skin contact distance of an individual of interest 118 which is an improvement over current health monitoring systems using radar signals. In some embodiments, when the health monitoring system 100 is not a wearable health monitoring system, such as when integrated or implemented within furniture or mounted to a wall, the health monitoring system 100 transmits and receives the electromagnetic signals as the individual of interest is within the skin contact distance of the health monitoring system 100. This may include when the individual of interest 118 is walking past the health monitoring system or sitting down on or proximate a piece of furniture where the health monitoring system 100 is integrated.

In some embodiments, system 100 may include more than one radar component 102 whereby each radar component 102 operates at a different frequency. In other embodiments, the radar component 102 may include two transmitters 102 operating at at least two non-overlapping frequency bands (for example around 2.45 GHz for lower frequency ranges and around 60 GHz in the mmWave range). The set of transmitters 102 may be designed to improve, increase or optimize tissue penetration and detection resolution by transmitting electromagnetic waves at the individual using different frequencies. The MCU 110 receives time-synchronized information or data that is collected from the at least one radar component 102 and may be used for data collection, synchronization, and signal processing. As discussed above, the processing of the signals may also be performed by the processor 112.

In one embodiment, the health monitoring system 100 collects data (such as in the form of signals and/or measurements) relating to, but not limited to, respiratory rate (RR), heart rate (HR), and heart rate variability (HRV) or that may then be processed to determine an individual's RR, HR and/or HRV.

In one embodiment, the radar component 102 operates using a frequency modulated continuous wave (FMCW) but may also use pulse width modulation (PWM).

In some embodiments, when the health monitoring system 100 is integrated within a wearable, the system 100 may include an adjustable ergonomic belt for fixing the health monitoring system 100 to a torso of the individual. In other embodiments, the health monitoring system 100 may be housed within a housing component that is attached to or part of the ergonomic belt. In yet other embodiments, the health monitoring system 100 may include a connector portion that mates with a corresponding connector portion attached to, or integrated with, the ergonomic belt. In some embodiments, the positioning of the health monitoring system 100 with respect to the individual is selected to allow it to rest at a preferred or optimal distance (which may or may not be the skin contact distance) from the individual's skin. Alternatively, the health monitoring system 100 may be integrated within other wearables, such as, but not limited, a watch, a ring or clothing.

One advantage of the disclosure is the provision of a health monitoring system 100 that is non-invasive such as when electrodes must be attached to an individual's body. In embodiments where the health monitoring system is integrated within a wearable, the disclosure provides a comfortable solution to obtain or provide on-going and regular measurements and/or results for health monitoring.

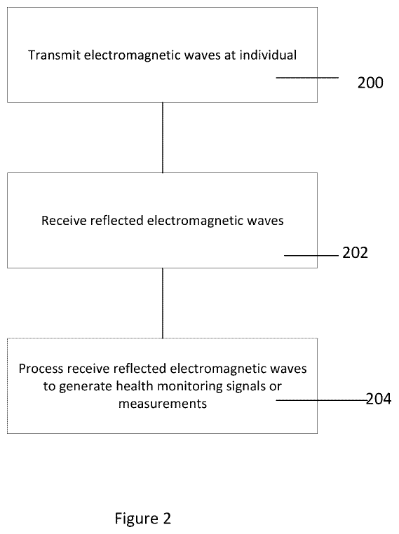

Turning to FIG. 2, a flowchart showing a method of health monitoring is shown. Initially, the health monitoring system transmits electromagnetic waves (such as via the set of transmitters) towards an individual (200). Any electromagnetic waves that reflect off the individual's skin are then captured by the health monitoring system (204), such as via the set of receivers. In some embodiments, depending on the location of the set of transmitters and the set of receivers, the electromagnetic waves that are received by the receivers may include deflected waves along with reflected electromagnetic waves.

The received reflected electromagnetic waves or signals are then processed (206) to calculate, generate or determine health monitoring signals or measurements. The health monitoring signals or measurements may include, but are not limited to, health bio-markers, cardio-vascular measurements and/or cardio-respiratory measurements and the like.

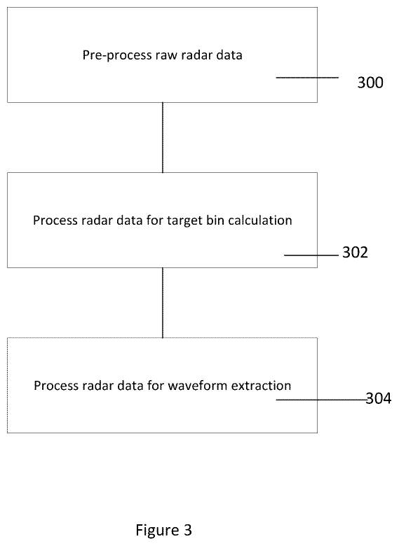

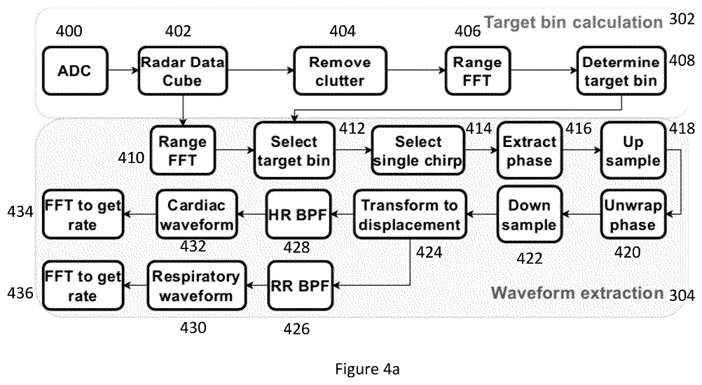

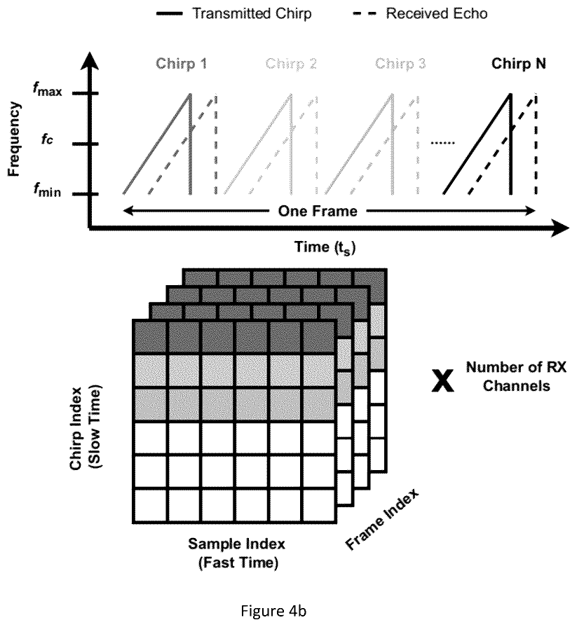

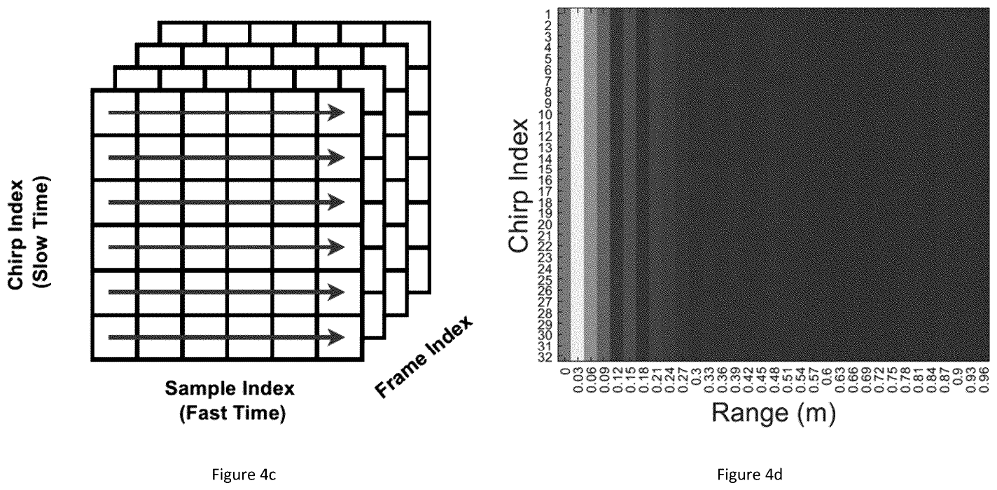

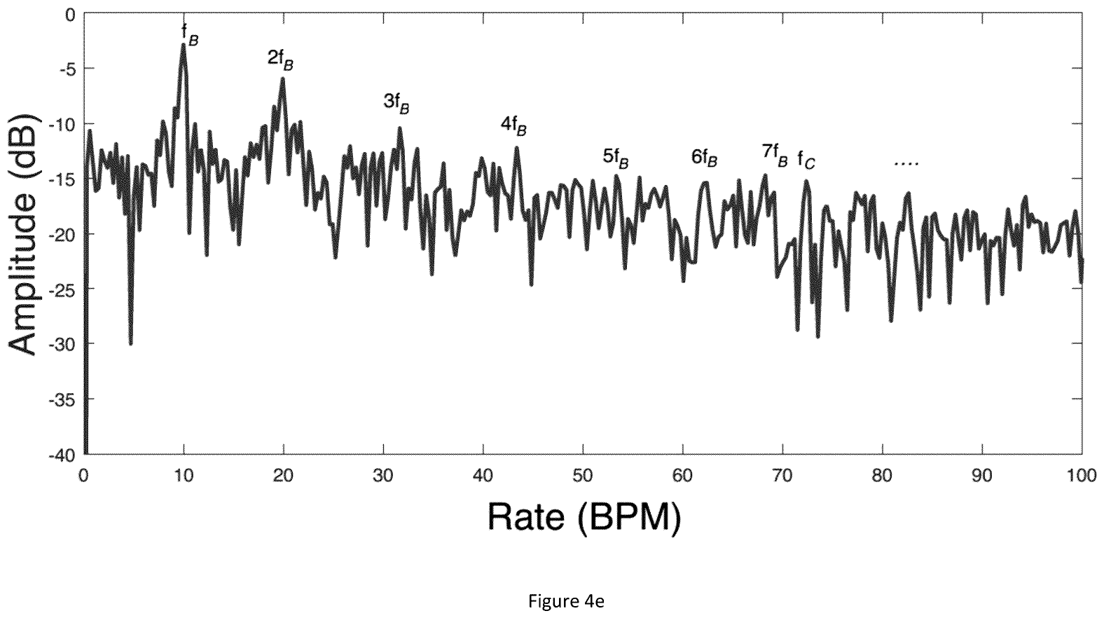

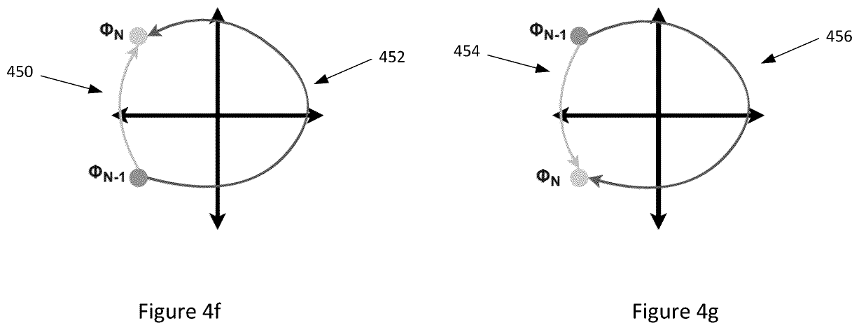

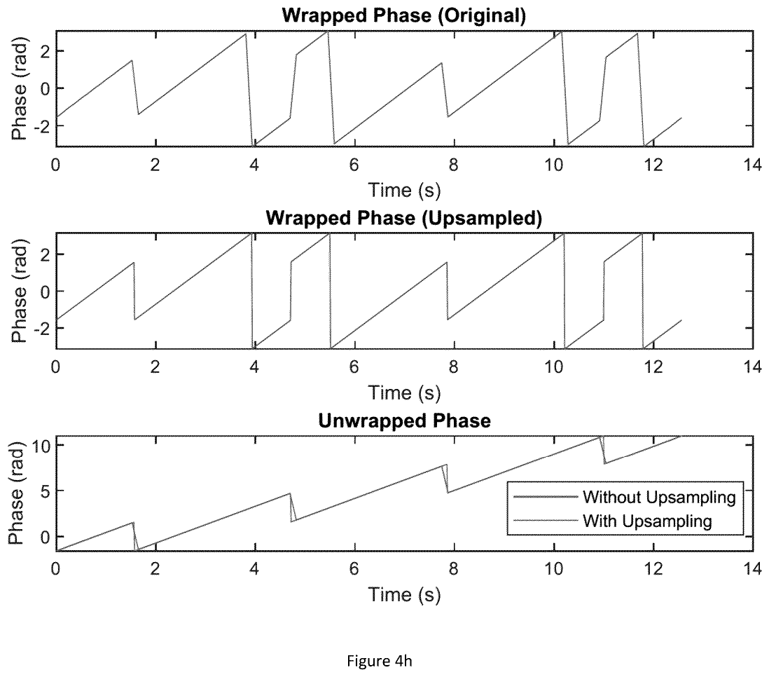

Turning to FIG. 3, a flowchart showing a method of processing the reflected signals to generate health monitoring, such as cardio-respiratory, measurements or signals is shown. In other words, FIG. 3 is directed at one embodiment of (204) of FIG. 2. In one embodiment, the reflected signals are processed to generate RR, HR and/or HRV signals. In the current embodiment, the reflected signals may be processed as part of a digital signal processing (DSP) chain to generate different components such as, but not limited to, a component for determining the target range bin and a component for extracting cardio-respiratory waveforms. One specific example of how to process the reflected electromagnetic waves is shown in FIG. 4a.

After receiving the reflected electromagnetic waves, the reflected electromagnetic waves or signals may be pre-processed (300) although this may not be necessary for all embodiments. The need to pre-process the set of reflected electromagnetic waves may be determined or predetermined by the set-up of the health monitoring system. The raw data can then be processed for target bin calculation (302) and waveform extraction (304).

In one embodiment for target bin calculation (as schematically shown in FIG. 4a), the reflected electromagnetic waves or the electromagnetic waves (or raw radar data) received by the set of receivers are down-converted (400). This may be performed using an analog-to digital converter (ADC). In a specific embodiment, the ADC is a 12-bit ADC and the down-converting is performed at about 1.0 MHz. After acquiring the down-converted raw radar data, represented as xb(t_{f}, tₛ), the down-converted raw radar data is organized into a radar data cube (402).

For the following specific example, the radar data cube is associated with a set of three channels as schematically shown in FIG. 4b. The radar data cube may be seen as a three-dimensional (3D) data structure with dimensions M×N×K, where M, N, and K are the number of chirps, samples, and frames, respectively. Processing of the different dimensions of the radar data cube assist to generate the health monitoring signals.

In order to more clearly understand the results or to determine a target range (such as a distance between the health monitoring system and a chest of the individual), the system then performs clutter removal (404) on the radar data cube. The clutter removal (404) removes unwanted extraneous echo related to the information stored in the radar data cube since this clutter can affect the acquired signal quality which interferes with the accurate detection and calculation of the health monitoring signals, such as cardio-respiratory rates, if not handled properly. Examples of clutter may include static clutter (caused by stationary objects in the environment); dynamic clutter (caused by moving objects within the health monitoring system's field of view) or multi-path reflections (caused by the reflected signals bounding off multiple surfaces before reaching the set of receivers).

In one specific embodiment of clutter removal, a mean of the raw signal across K frames is determined and subtracted from all M×N×K sample points in the cube. This may be performed by first discretizing the raw radar signal X_{beat}(t_{f}, tₛ) where:

$${x}_{beat} ( {t}_{f} , {t}_{s} ) \approx {A}_{TX} {A}_{RX} cos [ 4 π \frac{{R}_{0}}{{λ}_{max}} + 4 π \frac{Δ d ( {t}_{s} )}{{λ}_{max}} + 2 π {f}_{beat} {t}_{f} + {ϕ}_{T} ( {t}_{f} , {t}_{s} ) ] , τ < {t}_{f} < {T}_{c} \qquad Equation 1$$

with the result expressed as:

$${x}_{beat} [ n , m ] = {A}_{TX} {A}_{RX} cos [ 4 π \frac{{R}_{0}}{{λ}_{max}} + 4 π \frac{Δ d ( m )}{{λ}_{max}} + 2 π n {f}_{beat} + {ϕ}_{T} [ n , m ] ] \qquad Equation 2$$

$${θ}_{beat} = [ n , m ] = 4 π \frac{{R}_{0}}{{λ}_{max}} + 4 π \frac{Δ d ( m )}{{λ}_{max}} + 2 π n {f}_{beat} + {ϕ}_{T} [ n , m ] \qquad Equation 3$$

The discretized signal is then sampled at a sampling frequency where f_{beatmax}=1 MHz in the fast time and where t_{f}=n/f_{beatmax} and the signal is sampled at f_{c}=1/T_{c} in the slow time (i.e., tₛ=m/f_{c}). In the above equations, n and m represent fast and slow time indices from [0, N−1] and [0, M−1], respectively.

After clutter removal has been performed across multiple frames, the signal x_{b}[n,m] becomes y[n, m] such that

$$y [ n , m ] = {x}_{beat} [ n , m ] - \frac{1}{K} {\sum }_{k = 0}^{K - 1} {x}_{beat} [ n , m ] \qquad Equation 4$$

Once the set of reflected electromagnetic waves are processed such as to remove the clutter, a target detection is performed (406). In one embodiment, this may be seen as a range fast Fourier transform (FFT) as the FFT reveals range information. The range or distance between the health monitoring system and the chest of the individual, represented as R0, in each of the K frames, can be determined by applying a FFT across the N samples (i.e., across the fast time) for each of the M chirps (i.e., for each of the rows in the radar data cube) to obtain a spectrum of the beat signal. This spectrum will have a maximum, or high, peak indicating a position of the detected targets. Each frequency bin in the resulting spectrum corresponds to a particular distance at increments of the range resolution as specified in Equation 5 with a phase as described by Equation 3. This procedure is repeated for each of the K acquired frames.

$$\begin{matrix}⇒ {R}_{{0}_{min}} > \frac{c}{2 {T}_{c} K} \\ {R}_{{0}_{min}} > \frac{c}{2 B}\end{matrix} \qquad Equation 5$$

Looking at the signal after clutter removal along the fast time axis (i.e. along N), the FFT is used such that Y=F {y} for N samples as shown in Equation 6. In Equation 6, p is the index for the spectrum value for a given range bin within [(−N)/2, (N/2)+1].

$${Y}_{p} [ {ω}_{n} ] = {\sum }_{n = 0}^{N - 1} y [ n ] \cdot {W}_{n}^{np} , where {W}_{n} = exp ( \frac{- 2 π j}{N} ) is one of n roots of unity \qquad Equation 6$$

$${Y}_{p} [ {ω}_{n} ] = {\sum }_{n = 0}^{N - 1} y [ n ] \cdot exp ( \frac{- 2 π j}{N} np )$$

Since the signal sampled from the radar signal is real-valued, the resulting spectrum is conjugate symmetric (i.e., |Y [−ω]|=|Y [ω]| and ∠Y [−ω]=−Y [ω]), therefore the signal is sufficiently described by considering the spectrum in the range [0, (N/2)+1]. The result of the FFT is a complex sequence of values with magnitude and phase as represented by Equations 7 and 8. The angular frequency, ω=2 πf, is represented by Equation 9 since the sampling frequency is the limiting factor for the detectable beat frequency. The magnitude spectrum of the signal can be determined by applying Equation 7 to all points in [0, (N/2)+1], where the target bin with the maximum amplitude, argmax(□Yₚ[wₙ]□), indicates the range of distances (408) between the health monitoring system and the chest (seen as a chest range bin) as schematically shown in FIGS. 4c and 4d which schematically shown a range FFT operation on a radar cube dimension and a target position at 0.03, shown in one of the K frames, respectively.

$$\vert {Y}_{p} [ {ω}_{n} ] \vert = \sqrt{{ℜ𝔢 ( {Y}_{p} [ {ω}_{n} ] )}^{2} + {𝔍𝔪 ( {Y}_{p} [ {ω}_{n} ] )}^{2}} \qquad Equation 7$$

$${θ}_{{Y}_{p}} = {tan}^{- 1} ( \frac{𝔍𝔪 ( {Y}_{p} [ {ω}_{n} ] )}{ℜ𝔢 ( {Y}_{p} [ {ω}_{n} ] )} ) \qquad Equation 8$$

$${ω}_{n} = n \times \frac{{f}_{{beat}_{max}}}{N} \qquad Equation 9$$

Considering the previous analysis and the Nyquist sampling theorem, to detect a target at a range resulting in a given beat frequency (f_{beat}), the beat frequency (f_{beat}) must be greater than or equal to f_{beatmax}/2. Considering the round-trip delay time for signal propagation 2R₀ₘₐₓ/c, and the chirp slope K=B/T_{c}, the maximum or a highest detectable target range is described as R₀ₘₐₓ=(f_{beatmax}*c)/(2K). Likewise, a minimum resolvable beat frequency can be described as f_{beatmax}/N according to Equation 9, which can be written in terms of the minimum or low detectable range as R₀ₘᵢₙ=((f_{beatmax}/N)*c)/(2K)=c/(2B).

The system may then extract waveforms (304), such as in the form of cardio-respiratory signals, from the reflected signals (or radar data cube). For the extraction of cardio-respiratory signals, different measurements or calculations are performed depending on the desired health monitoring signal. For example, for target range selection, knowing the position of the chest R₀ of the individual (from target bin calculation (302)), it is possible to extract the vibrations due to the vital signs described as Δd(t) around the individual.

For the current example, since the methodology is based on displacement, and the radar component that was used is highly sensitive to the small vibrations of the chest, a single chirp is taken for all K frames. This eliminates or reduces the need to consider the M-length slow-time dimension in any further signal processing. More specifically, the sampling period of interest for observing phase difference due to the vibration of the chest is along the different frames, T_{R}.

This simplification reduces the size of the radar cube to an N×K matrix if all frames are processed at once to produce a single cardio-respiratory waveform and a signal reading for the respiratory rate and the heart rate. To create a continuous real-time estimate of the cardio-respiratory displacements and rates in time, a sliding window of a predefined length W along the K-length dimension across the period defined across the frames, T_{R}, can be used simplifying each iteration or to a N×W matrix.

For the waveform extraction, an FFT is applied (410) across the N-length dimension of the radar data cube (i.e., along the fast time) as schematically shown in FIG. 4c, producing a complex-valued spectrum of the radar signal. The result is a spectrum, where only the values in the range [0, (N/2)+1] are considered due to the conjugate symmetry of real-values raw data. The previously determined chest or target range bin (412) with the maximum or high amplitude, argmax(|Yₚ[ω]), is tracked across successive frames occurring at a period of T_{R}. For the specific experiment, this limits the experiment to a 1×W array (414) where W is a sliding window of predefined length with a maximum or high value equal to the number of frames, K.

With respect to phase extraction (416), the expression for the phase associated with the cardio-respiratory signal within one frame is listed above with respect to Equation 3. The vibration varies along the slow time in Equation 3. As tracking depends on the vibration across successive frames using only one chirp (i.e., one point in the slow time), tracking the phase expression in a given frame as represented by Equation 3 across different frames at a sampling period of T_{R} produces a signal with a frequency corresponding to the RR and HR. The envelope of this signal corresponds to the cardio-respiratory displacement of the chest.

A mathematical expression to simplify the visualization of the signal, assuming that the chest or target appears to be stationary to the radar can be generated, as the cardio-respiratory displacements are too small to be detected as range-bin variations. As such, the signal across K frames, sampled at a rate of f_{R} can be represented as a combination of the breathing signal with amplitude A_{B} and frequency f_{B}, the cardiac signal with amplitude AB and frequency f_{B}, and the total phase noise as shown in Equation 10.

$$ψ [ k ] = \frac{4 π}{{λ}_{max}} {A}_{B} cos ( 2 π \frac{{f}_{B}}{{f}_{R}} k ) + \frac{4 π}{{λ}_{max}} {A}_{C} cos ( 2 π \frac{{f}_{C}}{{f}_{R}} k ) + {ϕ}_{T} [ k ] \qquad Equation 10$$

Extracting the amplitudes enables the tracking of the chest displacement due to the heartbeat and respiration, and extracting the frequency components enables the extraction of the cardio-respiratory rates. For simplicity, Equation 10 may be seen as a simplification of the spectrum, which will be rich with many peaks at RR and HR harmonics, as well as their inter-modulation (IMD) products as schematically shown in FIG. 4e.

In some embodiments, arctangent demodulation (AD) can be chosen for phase analysis due to its relative insensitivity to local oscillator (LO) and mixer phase noise and its high sensitivity to small changes in displacement. The AD technique extracts the phase at the target range bin from the complex-valued FFT result according to Equation 8. The tan⁻¹ function produces outputs in the range [−π, π], and if the actual phase shift exceeds this range, the phase measurement wraps around and introduces a sudden discontinuity. Phase unwrapping (420) may be performed to remove these discontinuities to provide a continuous and accurate representation of the phase. Phase wrapping may include identifying the phase jumps or “wraps” and adding appropriate multiples of 2π to the phase values to ensure a smooth and unwrapped phase curve.

Detection of discontinuities requires knowledge of two successive phase values at a time, denoted by: ϕ_{N} and ϕ_{N-1}. A discontinuity occurs when |ϕ_{N}−ϕ_{N-1}|>π, and when this is true a phase correction must be applied to reveal the true phase value and remove any discontinuities:
where ϕ_{N}−ϕ_{N-1}>π indicates a clockwise jump across one quadrant but instead it is interpreted as a counter-clockwise jump across three quadrants without phase unwrapping due to the constraints of the range [−π, π] as schematically shown in FIG. 4f. In FIG. 4f, line 450 shows a correct path and line 452 shows a path before phase unwrapping. In this case, multiples of 2π must be subtracted from ϕ_{N}; and
where ϕ_{N}−ϕ_{N-1}<−π indicates a counter-clockwise jump across one quadrant, but is interpreted as a clockwise jump across three quadrants without phase unwrapping due to the constraints of the range [−π, π] as schematically shown in FIG. 4g. In FIG. 4g, line 454 shows a correct path and line 456 shows a path before phase unwrapping. In this case, multiples of 2π are added to ϕ_{N}.

In one embodiment, the system may perform up-sampling (418) on the radar data cube before phase unwrapping (420) in scenarios where the phase discontinuities are closely spaced, and the phase variations are small between adjacent samples. By increasing the sampling rate, up-sampling helps to capture finer details in the phase signal as schematically shown in FIG. 4h. This also improves the accuracy of the unwrapping process as outlined in Table 1 below.

TABLE 1

Algorithm 1 Phase unwrapping with up-sampling and down-sampling

Require: Phase sequence with phase wraps, ϕ[n]
Ensure: Unwrapped phase sequence, Φ[n]

| | |
|---|---|
| 1: | Up-sample ϕ[n] with interpolation to obtain \tilde{ϕ}[n] |
| 2: | Initialize Φ[0] = \tilde{ϕ}[0] |
| 3: | for n = 1 to N − 1 do |
| 4: | Δ\tilde{ϕ} = \tilde{ϕ}[n] − \tilde{ϕ}[n − 1] |
| 5: | if Δ\tilde{ϕ} > π then |
| 6: | Φ[n] = Φ[n − 1 − 2π |
| 7: | else if Δ\tilde{ϕ} < − π then |
| 8: | Φ[n] = Φ[n − 1] + 2π |
| 9: | else |
| 10: | Φ[n] = \tilde{ϕ}[n] |
| 11: | end if |
| 12: | end for |
| 13: | Down-sample Φ[n] with anti-aliasing LPF to obtain the final unwrapped phase sequence |

As will be understood, two scenarios that may benefit from up-sampling the data before unwrapping include high wrap density and small phase variations.

With high wrap density, when the phase wraps occur at a high density within a short interval, up-sampling can help resolve the phase ambiguities and provide a more accurate representation of the phase. By increasing the number of samples per wrap, up-sampling allows for better detection and unwrapping of phase jumps. With small phase variations, in cases where the phase variations between adjacent samples are small, up-sampling can enhance the resolution of the phase signal. This is beneficial when dealing with subtle phase changes or precise phase tracking whereby up-sampling increases the number of samples and improves the ability to detect and unwrap fine phase variations.

In order to maintain the phase information quality and to avoid artifacts introduced by the sampling rate conversion process, up-sampling is performed with interpolation techniques to estimate and insert additional samples by using mathematical functions to generate the intermediate data points. Interpolation preserves the signal's characteristics and captures the finer details introduced by the increased sampling rate.

Likewise, down-sampling (422) without proper filtering can introduce aliasing, which is the distortion of the signal due to overlapping frequency components. To prevent, reduce or remove aliasing, an anti-aliasing low-pass filter (LPF) may be applied before down-sampling to attenuate the high-frequency components in the signal. The filter ensures that the frequency content of the signal is limited to below the Nyquist frequency to avoid overlap and distortion.

With respect to rate extraction, the acquired 1×W array containing the phase, where W is a sliding window of some defined length with a maximum or high value equal to the number of K frames as previously mentioned, can now be transformed to a displacement array (424) as described by the expression for θ₁ in Equation 11:

$$θ ( {t}_{s} ) = 2 π {f}_{min} τ ( {t}_{s} ) = 4 π \frac{{R}_{0} + Δ d ( {t}_{s} )}{{λ}_{max}} \qquad Equation 11$$

$${θ}_{0} = 4 π \frac{{R}_{0}}{{λ}_{max}} , {θ}_{1} ( {t}_{s} ) = 4 π \frac{Δ d ( {t}_{s} )}{{λ}_{max}}$$

This waveform contains displacements due to both respiration and heartbeat signals as described in Equation 10. The signals are passed through a respiratory bandpass filter (426) and a heartbeat bandpass filter (428) resulting in a respiratory waveform (430) and a cardiac waveform (432), respectively. As mentioned previously, the HR typically occurs between 1-3 Hz (i.e., 60-90 beats per minute) and RR occurs at 0.1-0.5 Hz (i.e., 6-30 breaths per minute). Therefore, appropriate Butterworth FIR BPFs are used to separate the respiratory and cardiac waveforms from the spectrum shown in FIG. 4e. Keeping in mind that the amplitude of the cardiac displacement is much smaller than the amplitude due to the breathing displacement, the extraction of a detailed cardiac waveform (432) can be a challenge due to the potential overlap of high-order respiratory harmonics with the cardiac displacement waveform.

To establish a physiological basis for the extracted radar signals, in particular, for the complex cardiac displacement waveforms, the results were mapped against a ground-truth signal for cardiac monitoring such as an electrocardiogram (ECG). As described below, a simultaneously recorded ECG signal was synchronized and its features correlated to those extracted from a radar cardiac displacement waveform generated by the disclosure. Likewise, the RR, HR, and HRV extracted from the ECG signal were compared to those extracted from the cardiac displacement waveforms.

To extract the cardio-respiratory rates from the filtered chest displacement waveforms following the application of the proper filter, an FFT (434) is applied along the successive W-length displacement array windows. The frequency bin with the maximum amplitude, argmax(□Yₚ[ω]□), indicates the appropriate rate in Hz. This creates a continuous curve with time that tracks the biometrics of interest. By the Nyquist sampling theorem, the largest detectable frequency for this FFT would be f_{R}/2, which corresponds to about 10 Hz. This is more than sufficient for capturing the RR and HR. An FFT (436) can also be applied to the respiratory waveform to obtain respiratory rates.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding. For example, specific details are not provided as to whether elements of the embodiments described herein are implemented as a software routine, hardware circuit, firmware, or a combination thereof.

What is claimed is:

1. A method for health monitoring comprising:
transmitting electromagnetic waves at a target of interest;
receiving reflected electromagnetic waves from the target of interest; and
processing the reflected electromagnetic waves to determine health monitoring signals;
wherein processing the reflected electromagnetic waves includes performing a target bin calculation and extracting waveforms from the reflected electromagnetic waves.

2. The method of claim 1 wherein performing a target bin calculation comprises:
generating a radar data cube based on the reflected electromagnetic waves;
removing clutter from the radar data cube; and
applying a range Fast Fourier transform (FFT) to the radar data cube.

3. The method of claim 2 further comprising, before generating a radar data cube, processing the down-converting the reflected electromagnetic waves.

4. The method of claim 2 wherein extracting waveforms from the reflected electromagnetic waves comprises:
applying a range FFT to the radar data cube;
selecting a target range from the target bin calculation;
extracting phase from the radar data cube;
unwrapping phase from the radar data cube; and
transforming radar data cube to a displacement array to generate heart rate.

5. A system for health monitoring comprising:
a set of transmitters for transmitting electromagnetic waves toward an individual of interest;
a set of receivers for receiving electromagnetic waves reflected or deflected off the individual of interest; and
a processor for processing the reflected or deflected electromagnetic waves to determine a target bin calculation and to extract waveforms from the reflected or deflected electromagnetic waves.
